(19) United States
(12) Patent Application Publication
(10) Pub. No.: US 2013/0281885 A1
(43) Pub. Date: Oct. 24, 2013

(54) DEVICE WITH EXTERNAL PRESSURE SENSORS FOR ENHANCING PATIENT CARE AND METHODS OF USING SAME

(71) Applicant: Case Western Reserve University, Cleveland, OH (US)

(72) Inventors: James R. Rowbottom, Broadview Heights, OH (US); James Dixon Reynolds, Cleveland Heights, OH (US)

(21) Appl. No.: 13/916,840

(22) Filed: Jun. 13, 2013

Related U.S. Application Data
(63) Continuation of application No. PCT/US11/64587, filed on Dec. 13, 2011.
(60) Provisional application No. 61/422,364, filed on Dec. 13, 2010.

(51) Int. Cl. A61B 5/03; A61M 16/04; A61J 15/00; A61B 5/00

(52) U.S. Cl. CPC A61B 5/037; A61B 5/6853; A61M 16/0434; A61J 15/0003; A61B 5/447; A61M 2025/0002
USPC 600/587

(57) ABSTRACT

A method for monitoring physiologic conditions of a patient includes inserting an esophageal or other suitable tube into the patient, the tube comprising a lumen having a proximal end, a distal end, and central portion, and a pressure sensor disposed about at least a portion of an outer surface of the lumen of the tube. The pressure sensor is capable of detecting a change in pressure when a force is exerted against the outer surface of the lumen. The tube is then positioned within the patient and an initial pressure reading of the pressure exerted against the pressure sensors disposed about the at least a portion of the outer surface of the lumen of the tube is taken. The method also includes monitoring changes in the initial pressure reading and, if needed, taking a second pressure reading.

### RELATED APPLICATIONS

This application claims priority to International Application No. PCT/US11/64587, filed on Dec. 13, 2011, entitled DEVICE WITH EXTERNAL PRESSURE SENSORS FOR ENHANCING PATIENT CARE AND METHODS OF USING SAME, which claims priority to U.S. Provisional Patent Application No. 61/422,364, filed on Dec. 13, 2010, entitled APPARATUS AND METHODS FOR EVALUATION OF THE POST INTUBATION AIRWAY AND READINESS FOR AND SAFETY OF TRACHEAL EXTUBATION.

### FIELD OF THE INVENTION

This application relates in general to a device and method for reliably monitoring the physical conditions of a patient. Specifically, the device includes the use of a tube-like or other suitably shaped structure with external pressure sensors that, when inserted into or on the patient, enables a physician or other care-giver to monitor the pressure exerted by the patient against the structure and to extrapolate various physical conditions of the patient.

### BACKGROUND OF INVENTION

Tubes, such as endotracheal or feeding tubes, are often placed into patients to help them breathe, provide nutrition, administer drugs, etc. Despite great attention to safety, these tubes can be placed in the wrong position or can move from the desired position during the course of therapy. Being able to readily confirm tube position is particularly important with endotracheal and feeding tubes where incorrect placement of the tube can have catastrophic results. In addition, despite the invasiveness of tube placement little effort is directed at leveraging the tube's position to collect physiologic data on patient status or to provide information to the caregiver when it is safe to remove the tube. This latter point is particularly true for intubation of the trachea where tube placement may result in injury to the airway, vocal cords and tracheal mucosa due to excessive cuff pressure or oversized endotracheal tubes (ETT). It is also true for when procedures are conducted on the neck that can produce swelling that exerts pressure on the airway.

Endotracheal tubes (ETT) may be placed in a trachea to provide controlled ventilation of (and administration of anesthesia to) humans and animals undergoing medical procedures. Significant attention has been focused on safety of the intubation procedure as well as ETT design features and processes to minimize injury to the airway. Despite these measures, vocal cord and tracheal mucosal injuries still occur. Some of the injuries are the result of mucosal ischemia from increased ETT cuff pressure or possibly oversized ETT's. Conversely, there has been little attention focused on extubation of the difficult airway which can be the source of significant patient morbidity and mortality, especially in high risk populations. There are multiple factors involved in the decision to remove the ETT but this action is based on one premise: the patient has a patent airway and can breathe on their own. Airway patency is assessed using the cuff-leak test. The cuff-leak test is performed by deflating the ETT cuff while the patient is spontaneously breathing. Once deflated, the ETT is occluded with the physician's (or veterinarian's) thumb and the patient is observed for signs of effective breathing, including respiratory effort, chest rise, and, most importantly, the audible inspiratory and expiratory breath sounds at the level of the oropharynx.

Many of the signs of effective breathing that are used to determine the patency of the patient's airways are subjective. It would be beneficial to provide the physician (or veterinarian) with objective or qualitative indicators, such as a device or method, to assess the patency of the airway, to objectively define the post intubation airway, create an objective and reproducible cuff-leak test ultimately contributing to safer airway management in both operative and non-operative settings.

### SUMMARY OF THE INVENTION

A system for monitoring physiologic conditions of a patient includes a medical device having an outer surface and a pressure sensor disposed about at least a portion of the outer surface of the medical device, wherein the pressure sensor is capable of detecting a change in pressure when a force is exerted against the outer surface of the medical device. The medical device may be an endotracheal tube, an esophageal feeding tube, a catheter, or a dermal pad.

In one embodiment, the medical device is an endotracheal tube having a lumen with a distal end, a central portion, and a proximal end. The endotracheal tube may further include an inflatable cuff disposed between the central portion of the lumen and the distal end of the lumen. In this embodiment, the pressure sensor may be disposed on the outer surface of the central portion of the lumen and/or the outer surface of the distal end of the lumen. The pressure sensor may also be integral with the central portion and/or the distal end of the lumen. The pressure sensor may be a force sensing resistor, a mesh of micro-wires, a pressure sensitive conductive polymer or other biocompatible material, or a combination thereof.

In another embodiment, a method for monitoring physiologic conditions of a patient, includes inserting an endotracheal tube through a mouth of the patient, the endotracheal tube comprising a lumen having a proximal end, a distal end, and central portion and an inflatable cuff disposed around the lumen, between the central portion of the lumen and the distal end of the lumen, and a pressure sensor disposed about at least a portion of an outer surface of the lumen of the endotracheal tube; wherein the pressure sensor is capable of detecting a change in pressure when a force is exerted against the outer surface of the lumen; positioning the endotracheal tube within the patient's trachea so that the patient may breath through the endotracheal tube; taking an initial pressure reading of the pressure exerted against the pressure sensors disposed about the at least a portion of the outer surface of the lumen of the endotracheal tube; and monitoring changes from the initial pressure reading.

The method may also include the step of taking at least a second pressure reading and detecting movement of the endotracheal tube by comparing the initial pressure reading with the second pressure reading and determining if the endotracheal tube has changed positions.

In another embodiment, the method further includes the step of evaluating the patency of a patient's airway by comparing the initial pressure reading with the second pressure reading and determining if tissue around the endotracheal tube has swollen.

In yet another embodiment, a method for monitoring physiologic conditions of a patient includes inserting an esophageal feeding tube through the nose or mouth of the patient, the esophageal feeding tube comprising a lumen having a proximal end, a distal end, and central portion, and a pressure sensor disposed about at least a portion of an outer surface of the lumen of the esophageal feeding tube; wherein the pressure sensor is capable of detecting a change in pressure when a force is exerted against the outer surface of the lumen; positioning the esophageal feeding tube within the patient's esophagus; taking an initial pressure reading of the pressure exerted against the pressure sensors disposed about the at least a portion of the outer surface of the lumen of the esophageal feeding tube; and monitoring changes from the initial pressure reading. The method may also include monitoring the placement of the esophageal feeding tube within the esophagus by comparing the initial pressure reading with a known pressure pattern for a patient's esophagus.

### DESCRIPTION OF THE DRAWINGS

FIG. 1 is a perspective view of one embodiment of an endotracheal tube.

FIG. 2 is a depiction of an endotracheal tube positioned within a human body.

FIG. 3 is a depiction of a pressure sensor for use with an endotracheal tube.

FIGS. 4a and b are orthogonal polarization spectral images of a subject's sub-lingual micro-vasculature.

FIG. 5 is a perspective view of an airway management system.

FIG. 6 is a perspective view of an endotracheal tube.

FIG. 7 is a perspective view of a display unit for use with an endotracheal tube.

FIGS. 8a and b are depictions of endotracheal tubes positioned within a human body.

FIG. 9 is a perspective view of a display unit for use with an endotracheal tube.

FIG. 10 is a perspective view of one embodiment of a feeding tube.

FIG. 11 is a perspective view of one embodiment of a dermal pad.

### DETAILED DESCRIPTION

A system for enhancing patient care includes an external sensor and may be used to reliably monitor the physical conditions of a patient. The system may include a medical device having an outer surface and at least one pressure sensor disposed about at least a portion of the outer surface of the medical device. The pressure sensor is capable of detecting a change in pressure when a force is exerted against the outer surface of the medical device. The medical device may be any suitable device, such as an endotracheal tube, an esophageal feeding tube, a catheter, or a dermal pad.

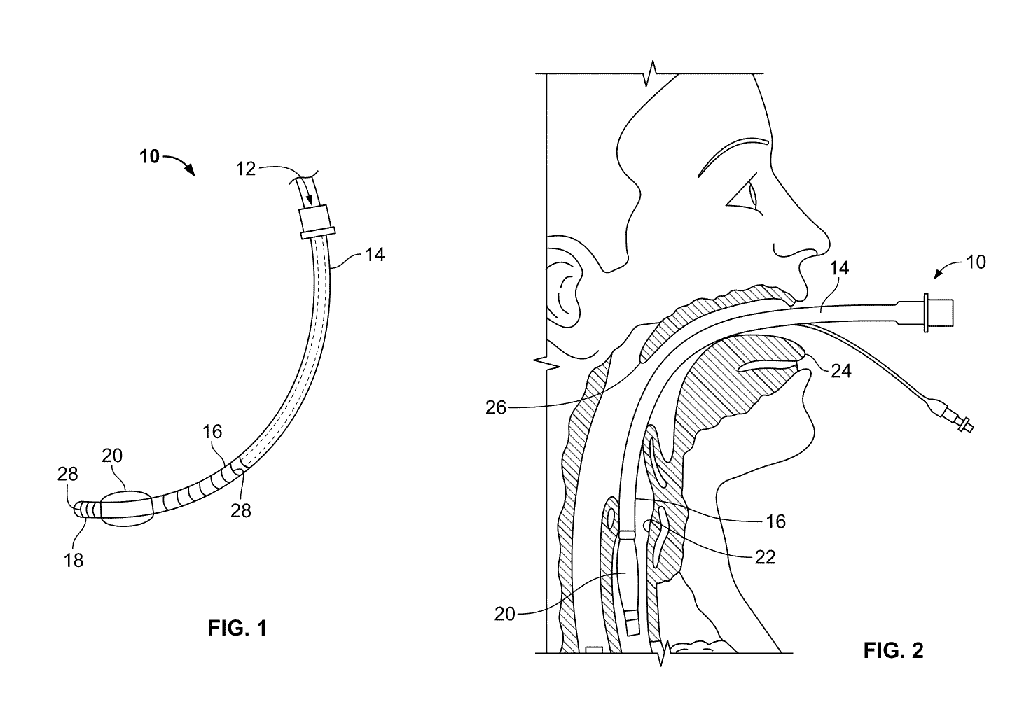

In one embodiment, as shown in FIG. 1, the device may be an endotracheal tube (ETT) 10. The ETT 10 includes a rapid means to reliably monitor intubated patients in the intra-operative, post-operative, critical care, military, resuscitative, and transport environments. The ETT 10 includes a lumen 12 with a proximal end 14, a center portion 16, and a distal end 18. The ETT 10 may also include an inflatable cuff 20 disposed around the lumen 12 of the ETT 10 between the distal end 18 and the central portion 16. The cuff 20 may be designed to expand at least a portion of an outer circumference of the lumen 12, preventing the flow of air to and from the patient's lungs via the space between the outer surface of the lumen 12 and the patient's trachea. When inflated, the sole pathway for air to pass into the patient lungs is through the lumen 12 of the ETT 10, rather than around.

As shown in FIG. 2, when the ETT 10 is inserted into a patient's trachea, the inflatable cuff 20 is generally disposed just below the patient's vocal cords 22 and the proximal end 14 of the lumen 12 extends into and beyond the patient's mouth 24, with at least a portion of the proximal end 14 of the lumen 12 remaining outside of the body. In this embodiment, the central portion 16 of the lumen 12 is disposed in the trachea of the patient, between the patient's vocal cords 22 and the patient's soft palate or uvula 26.

Referring again to FIG. 1, the central portion 16, the distal end 18, and/or the cuff 20, of the lumen 12 may include a pressure sensor 28 disposed around the exterior of the lumen 12. The pressure sensor 28 may generally extend along a substantial portion of the central portion 16 and distal end 18 of the lumen 12. In one embodiment, the pressure sensor 28 extends from the back of the patient's mouth 24 to approximately the middle of the trachea. In another embodiment, the pressure sensor 28 may also cover the inflatable cuff 20 and/or the distal end 18 of the lumen 12.

The pressure sensor 28 may be separate from or integral with the lumen. The pressure sensor 28 may comprise any suitable, biocompatible, sensor that does not significantly increase the outer diameter of the lumen 12. The pressure sensor 28 may include force-sensing resistors (FSR), such as, among other suitable sensors, the NPC-100 or NPM-100, both available from General Electric, the Interlink Electronics Force Sensing Strip, and FlexiForce sensors available from Tekscan, including models A201-1, A201-25, and A201-100. The sensors 28 may include microelectromechanical and/or malleable systems and sensors.

The force-sensing strip, for example, measures the force applied on the sensing area by varying the resistance. As the force applied increases, the resistance of the strip changes, which can be used to determine the value of the force exerted against the pressure sensor 28.

As shown in FIG. 1, the force-sensing strip may be wrapped around the central portion 16 and/or the distal end 18 of the lumen 12 and coated with a medical grade gel to prevent water and other bodily fluids from affecting the functionality of the pressure sensor 28. Generally, the pressure sensor 28 will remain accurate while in the mouth 24 and tracheal environment for up to three weeks.

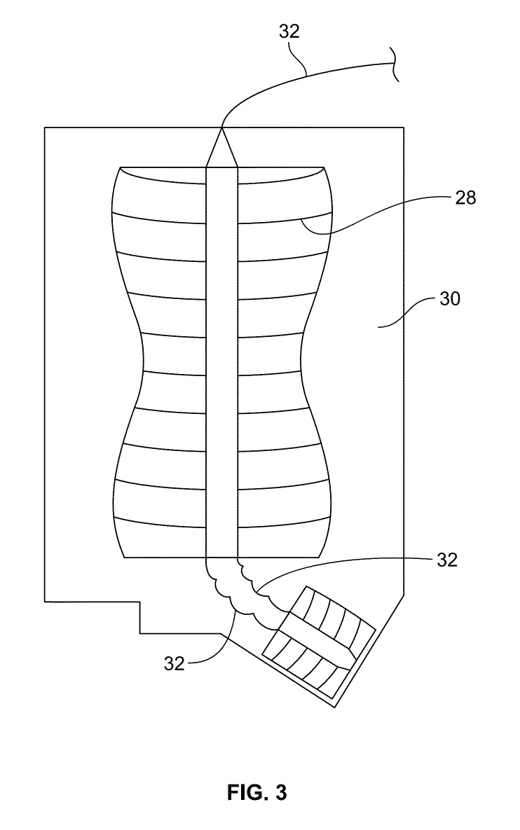

As shown in FIG. 3, the pressure sensors 28 may be incorporated into a sheet of adhesive film 30 and applied to a standard endotracheal tube. In one embodiment, the sheet of adhesive film 30 may have an hour glass shape, as shown in FIG. 3, or any other shape that is best suited to cover the central portion 16 of the lumen 12. Moreover, such pressure sensors may also be similarly adhered to the distal end of the lumen 12 that extends beyond the cuff so that pressure may similarly be measured above and below the cuff. The sensors located above and below the cuff may be electronically attached to one another using electronic leads 32 so that the pressure readings may be easily compared and used to diagnose changes in the patient's airway. Circuits for the FSRs can be printed with a high density (100 s per inch) that allows for very fine resolution of the pressure pattern exerted upon the ETT.

In yet another embodiment, the pressure sensor 28 may be integral with the lumen 12. In this embodiment, the lumen 12, or at least a portion of the lumen 12, is formed of a material with conductive properties, such as polyurethane. One suitable polyurethane, developed by Cleveland Medical Polymers in Medina, Ohio, includes a series of pressure sensitive carbon nanotubes (not shown). The conductive polymer (CPU), such as polyurethane, has electrical properties that change upon application of mechanical strain (i.e. pressure). CPU maintains the electro-responsive property even as it is molded into complex shapes, which allows for the placement of sensors into highly constrained geometries.

In addition, should the CPU be adhered to the outside of an already formed lumen 12, the electro-responsive property is maintained even with thin layers of CPU, approximately less than 0.5 mm in thickness, such that extern application to an ETT or other such tube will result in only a very minimal increase in overall diameter.

When using a conductive polymer, a series of interconnected micro-wires may be implanted into the CPU by sandwiching the wires between two thin sheets of CPU to create a broad array of discrete pressure sensors 28, i.e. to create a pressure footprint along the length of the lumen 12. It should be noted that the pressure footprint may also be created using the force-resistant sensors, described above or other force sensing biocompatible material.

The pressure-sensing array may next be attached to the outer surface of the ETT 10 to circumscribe the parts of the lumen that would be in contact with the sections of the human (or animal) respiratory track following intubation. Spontaneous contraction of the human vocal cords exerts pressures of 5 mm Hg for 1 to 2 seconds; pressures generated during activity (e.g. swallowing) can be 20-fold higher.

The CPU serves as a piezoelectric-conducting material with conductance inversely proportional to its resistance. Incorporation of the wire-mesh assembly into the CPU creates an array of variable resistors where pressure-induced deformations result in local changes in conductivity. A constant current is driven across the variable resistors (sensors). As the polymer deforms in response to pressure there is a change in voltage that correlates to the force applied and it is localized to the site of application; multiple pressure points or pressure applied over a large area will produce discrete changes in voltage output from each “pressure sensor” (i.e. resistor). The voltage signal will be processed through a specialized multi-input microcontroller. A corresponding monitor may be used to monitor the pressure readings and may include multiple analog input ports and outputs.

The microcontroller may sample voltages at the various rates as required by the embedded processing code. This code may include a module that filters out power line and all unwanted high/low frequency noise using Chebyshev or other appropriate digital filtering techniques. The code may output a correlated pressure reading to each voltage drop at the input. Additionally, the microcontroller requires voltage for operation, which includes driving the current through the CPU/wire-mesh assembly.

In one embodiment, the pressure footprint may be used to ensure proper placement of the ETT 10 relative to the patient's vocal cords. The presence of the tip of the ETT 10 between the cords on a “difficult or blind” intubation serves to guide insertion safely while more proximal pressure sensors residing between the vocal cords serves to monitor and confirm continued and correct positioning between the vocal cords.

In one embodiment, the ETT 10, as shown in FIG. 1, may be placed in the body of the patient before a patient undergoes surgery so that the distal end 18 of the lumen 12 is disposed at or below the patient's vocal cords 22. The inflatable cuff 20 should be inflated, effectively sealing off of the patient's airway, except for through the lumen 12 of the ETT 10. Using the display unit (not shown), the pressure sensor 28 measures the pressure exerted on the lumen 12 of the ETT 10 under pre-surgical conditions, presumably before swelling has occurred. During and after surgery, the pressure exerted against the lumen 12 of the ETT 10 is measured continuously or intermittently. The physician may use the pre- and post-surgical pressure levels as an objective indicator of the patient's ability to breath without aid of the ETT 10 and that the patient's airway is not likely to swell closed once the ETT 10 is removed.

In another embodiment, the ETT 10 may include carbon dioxide (CO₂) sensors (not shown) that are placed on the outside of the lumen 12 of the ETT 10 at two points along the distal length of lumen 12, specifically above and below the cuff. Following cuff deflation the CO₂ sensors will record changes in CO₂ as the patient breathes. Supraglottic edema or other airway obstruction would be identified by discordance in the two CO₂ values, i.e. the lower sensor values would increase reflecting the accumulation of CO₂ in the lungs while the readings from the upper sensor would decrease as the amount of expired air flowing across the band declines. In one embodiment, the CO₂ sensor may include a fluoropolymer microfilm or opto-chemical technology to ensure the CO₂ sensors around the ETT 10 are unobtrusive so as not to impact intubation. Other embodiments of the ETT 10 may include heart rate, blood pressure, pulse oximetry, near-infrared or visible light spectroscopy, lactate, and pH monitors placed on the lumen 12 of the ETT 10.

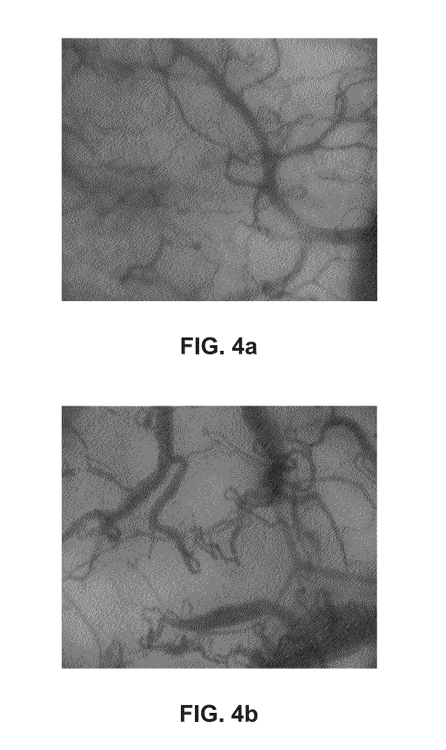

The ETT may also be used to monitor and assess microvascular flow. As shown in FIGS. 4a and 4b, orthogonal polarization spectroscopy images of the sub-lingual micro-vasculature were obtained under normoxic (FIG. 4a) and hypoxic (FIG. 4b) conditions. Sidestream dark field imaging may be used to also directly evaluate microvascular networks. It is noted that under hypoxic conditions the number of visible small vessels is considerably decreased as the movement of red blood cells declines. Therefore, a sensors placed along the proximal portion 14 of the ETT 10 may allow a physician to monitor the blood flow through the microvasculature at the base of the tongue or other microvascular networks in the airway covered by a thin epithelium. Should the patient become deprived of oxygen, the number of visible small vessels in the monitored region would decrease, alerting the physician immediately and adjust the ETT or other treatment accordingly.

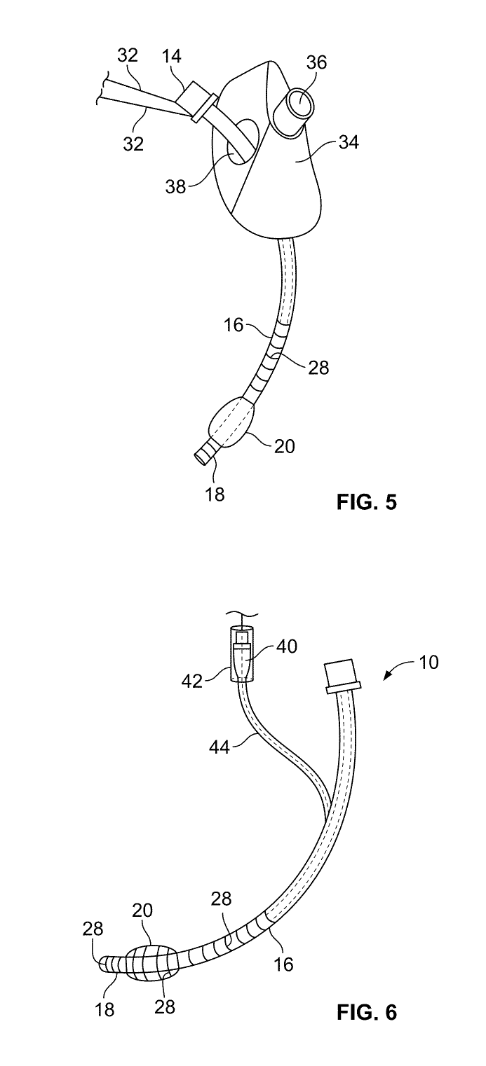

As shown in FIG. 5, the ETT 10 may also include a mask 34. The mask 34 is pre-connected to a gas line 36 that detects carbon dioxide concentration. The physician detaches the ventilator from ETT 10 at the proximal end 14 of the lumen 12 and places the mask 34 over patient's airway while threading the lumen 12 through an airtight side hole 38 in the mask 34.

In this embodiment, the proximal end 14 of the lumen 12 is capped so that all of the exhaled air from around the external surface of the lumen 12 goes out of the mask outlet 36 and the CO₂ concentration is displayed on a display unit. In this manner, physicians have yet another way to make the cuff-leak test more objective in order to assess the patency of the patient's airway. The amount of carbon dioxide flowing around the occluded lumen 12 of the ETT 10 is indicative of how well the patient is able to breath on their own.

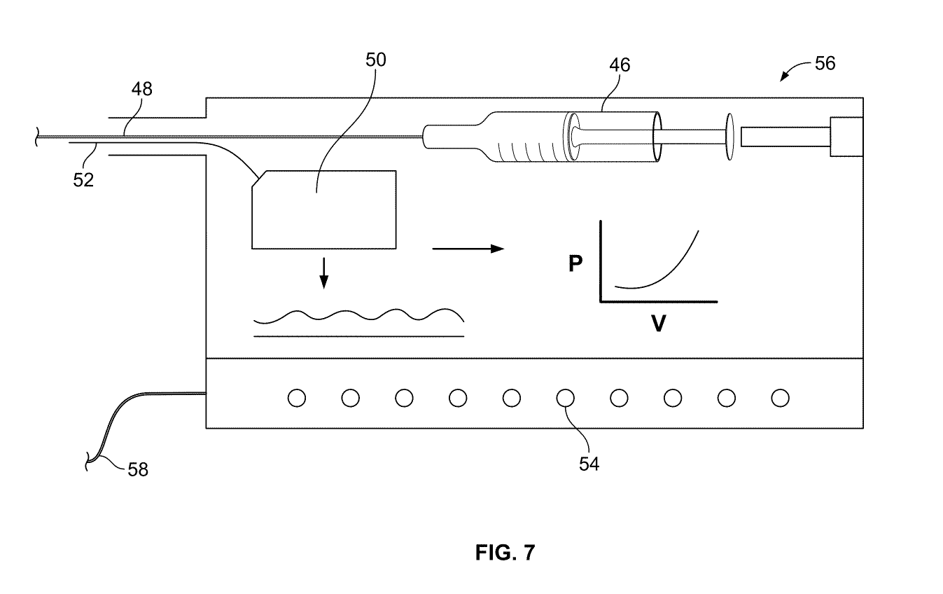

In another embodiment, as shown in FIG. 6, the ETT 10 may also include a mechanism that allows the physician to interpret pressure changes inside the inflated cuff. In this embodiment, the ETT 10 may include a pilot balloon 40 with an outer rigid sleeve 42 and inflation tube 44 attaching the pilot balloon 40 to the central portion 16 of the lumen 12. As shown in FIG. 6, the cuff 20 may be rapidly inflated using an automatic syringe 46 that inflates the cuff 20, through a first channel 48 (as shown in FIG. 7), wherein the pressure within the inflated cuff 20 is measured, by measuring the pressure within the pilot balloon 40, with a pressure transducer 50 through a second channel 52, moderated with a stopcock valve.

When the cuff 20 is fully inflated, the cuff 20 will press against the side wall of the patient's airway and the pressure within the pilot balloon 40 will increase dramatically, indicating to a physician that the cuff 20 is fully inflated, and thus providing an objective indication that the ETT 10 is properly sized and fitted to the individual patient. Moreover, the automatic syringe 46, the first 48 and second channels 52, the pressure transducer 50, and indicator lights 54 indicating the pressure level readings from the sensors 28 may all be incorporated into one display 56. Alternatively, the fit of the ETT 10 in the patient's airway may be measured by calculating the change in pressure exerted against the surface of the patient's airway by using pressure sensors 28 disposed on the outer surface of the cuff 20 to determine when the cuff 20 is fully inflated.

In addition to measuring fit of the ETT 10 in the patient's airway, if a minimal amount of air is left in a deflated cuff 20, the pressure within the cuff 20 may also be measured by the pressure transducer 50 to assess if a patient is having trouble breathing on their own around the deflated cuff 20 and occluded ETT 10. Using the ETT 10 to detect supraglottic edema (change in pressure against the ETT over time, or more importantly—resolution of changes indicating enhanced safety of removing the ETT 10), detect air movement past the occluded ETT 10 with cuff 20 minimally inflated and transduced may be used as the foundation of an objective cuff-leak test to assess airway patency prior to extubation.

The display unit 56 may be connected either with wires or wirelessly to the pressure sensor 28 on the lumen 12, carbon dioxide sensors, the pressure transducer, or any other suitable indicator included on the ETT 10. Moreover, the display unit and the ETT may be monitored and controlled remotely, such as with a remote control or with an application for a remote device, such as a smart phone or home computer. This may be done using Bluetooth, Wi-Fi, 3G, or RF wireless communication methods. It is contemplated that other suitable wireless communication methods may also be used. The information may also be integrated into the patient's electronic medical records.

In one embodiment, the display unit 56 may be small enough to be held and operated in the hand of the physician. In this embodiment, the pressure sensor 28 may be connected to the display unit 56 by a JST connecter 58 and may consist of an electronic enclosure including a microcontroller, such as the Arduino Duemilanove. The display unit 56 may also include a multi-character and segment display and may incorporate light emitting diodes 54 or other suitable visual displays. The display unit 56 may also be powered by a portable energy source, such as batteries or may be plugged in to a traditional power source. The display unit 56 may be part of a larger operating suit or electronic interface and may have electronic data storage capabilities.

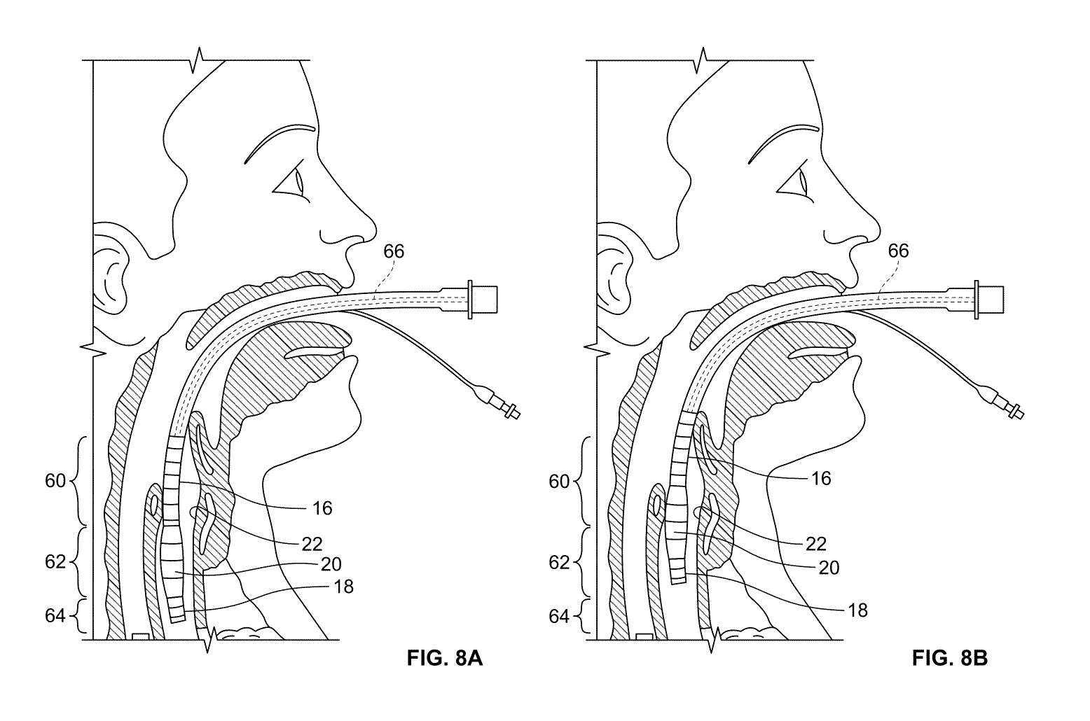

As shown in FIGS. 8a and 8B, the physician may use the pressure sensors 28 to monitor the position of the ETT 10 in the patient's body. Monitoring the position of the ETT is important to preventing damage to the patient's vocal cords and to warn of unplanned tube movement or impending tube expulsion. For example, if the inflatable cuff 20 is in contact with the patient's vocal cords, it may cause granulomas, polyps, and/or ischemic damage. Currently, the position of an inserted endotracheal tube (ETT) is monitored using a daily X-ray. This method, however, may actually cause movement of the ETT, so obtaining an accurate measurement may prove difficult.

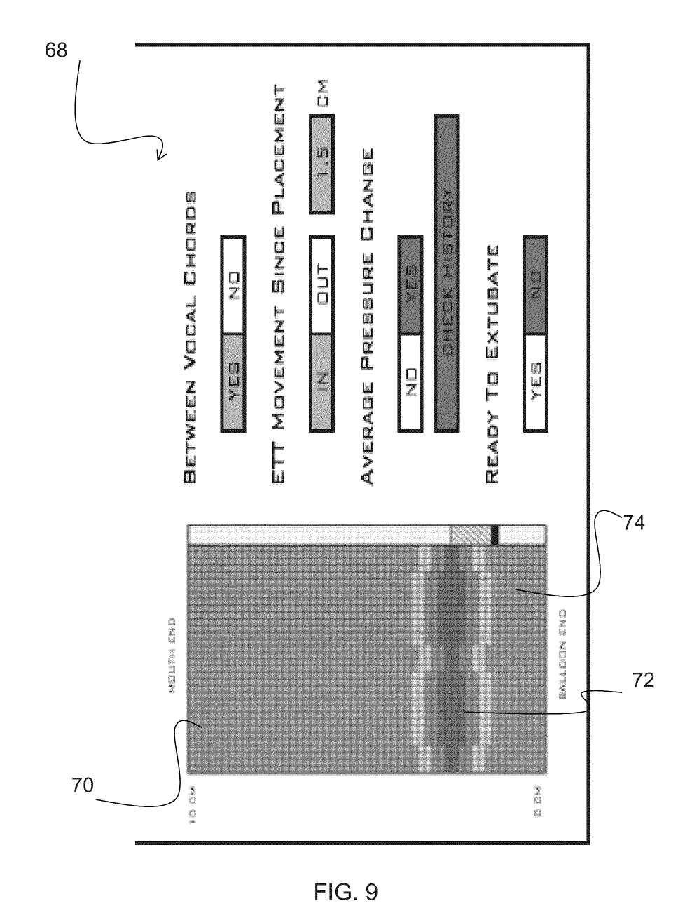

In this embodiment, the pressure sensors 28 cover the central portion 16 and distal end 18 of the lumen 12 and/or the inflatable cuff 20. The pressure sensors 28 may be comprised of force sensing strips, a sheet of adhesive film, CPU, or other conductive biocompatible material as described above. In order to monitor the position of the ETT 10 within the body, shortly after the ETT 10 is inserted into the patient's trachea at a desired location (immediately after insertion of the ETT), the physician obtains initial pressure readings along portion of the ETT 10 covered with pressure sensors 28, creating an initial pressure footprint. That footprint is recorded or transmitted electronically, either by using wires 66 or wirelessly, into a display unit 58, as shown in FIG. 9.

The regions of the patient's airway exerting pressure against the portions of the ETT 10 being monitored by the pressure sensors 28, i.e. the central portion 16, the distal portion 18, and/or the inflatable cuff 20, may be assigned number ranges or colors on a graphical output corresponding to the initial pressure footprint. For example, a first region of the airway 60 may be assigned the color green 70 and may correspond to the pressures exerted against the central portion 16 of the lumen. A second region of the airway 62 may be assigned the color red 72 and may correspond to the pressures exerted against the inflatable cuff 20. A third region 64 may be assigned the color yellow 74 and may correspond to the pressures exerted against the distal portion 18 of the lumen. Alternatively, the colors may be assigned based upon intensity of pressure readings against the pressure sensors 28 or, once establishing the optimal position of depth, the position of the ETT 10 relative to the vocal cords.

In the embodiment where the colors are assigned based upon the position of the ETT 10 relative to the vocal cords, the vocal cords would be the reference point from which a “green” zone would be established on the display representing 2 cm above and below the reference; the “yellow zone” would be displayed as 2-3 cm above and below the reference and the “red zone” would represent movement of the ETT 10 greater than 3 cm above or below the established reference point—with a corresponding alarm alerting the clinician to dangerous movement or misplacement.

Generally, the first region 60 and second region 62 of the airway should be demarcated by the location of the vocal cords relative to the ETT. This will provide a reference point to the physician. Other reference points within the body, however, may be used.

Because the cuff 20 is inflated to a diameter larger than that of the central or distal portions of the lumen, the pressure exerted against the cuff 20 may likely be different than the pressure exerted against the rest of the ETT 10. Therefore, if the ETT 10 should move, as shown in FIG. 9b for example, so that the inflatable cuff 20 is positioned closer to or past the vocal cords 22, the pressure reading taken for the respective regions of the airway would likely change, indicating to the physician that at least a portion of the cuff 20, for example, had moved from the red region (below the vocal cords 22) to the green region (above the vocal cords). Therefore, rather than monitor the location of the ETT 10 with an x-ray machine, the physician or his assistant may constantly monitor the position of the ETT throughout the day without moving or disturbing the patient. And, if using a remote access, such as Wi-Fi, the physician can monitor the position of the ETT from virtually anywhere.

It should be appreciated that the colors used and the number of regions demarcated in the patient's trachea may vary, depending on the preference of the physician and the purpose of monitoring the pressures and location of the ETT. For example, the pressure around the vocal cords can be monitored to determine when the muscle relaxant, given to the patient upon intubation, is beginning to wear off.

Additional benefits of the pressure sensing functionality of ETT include, but are not limited to assessment of swallowing, aspiration risk, assessment of vocal cord movement to detect recurrent laryngeal nerve injury, assessment of return of vocal cord movement during general anesthesia with muscle relaxants indicating time for redosing, non-radiographic representation of ETT position/location in the airway to detect changes with patient movement (ICU patients), identifying mainstem intubation or impending extubation, indicate position of individual lumens of a double lumen endotracheal tube, decreased radiation exposure to long term intubated ICU patients, and tissue oxygenation sensing, and/or microvasculature blood flow imaging to minimize and/or diagnose tissue ischemia.

It should also be appreciated that the inventions described for use with an endotracheal tube may also be used with esophageal feed tubes, Foley catheters, to continuously monitor bladder pressures as a reflection of intra-abdominal compartment pressures or other medically necessary tubes to monitor pressures within the patient and improve the quality of care. Such tubes may also include heart rate, blood pressure, pulse oximetry, near-infrared or visible light spectroscopy, lactate, and pH monitors placed on the lumen. For example, a hemoglobin sensor on a feeding tube or Foley catheter could be used to provide early warning of GI bleeding or of the presence of blood in the urine thus prompting additional intervention by the physician, veterinarian, or other type of caregiver.

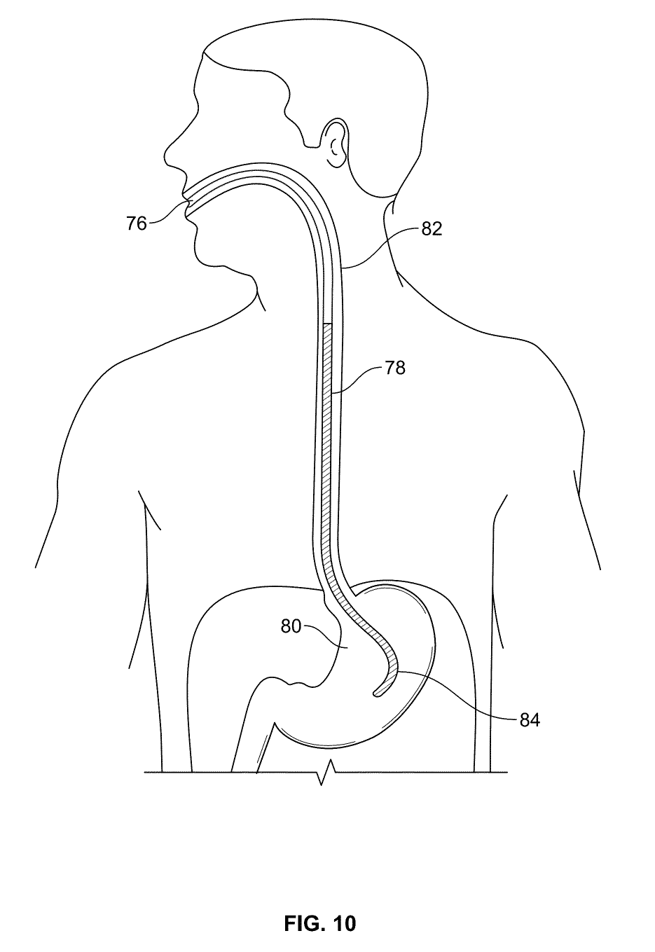

As shown in FIG. 10. in another embodiment, the system may include a feeding tube 76 with pressure sensors 78 disposed along at least a portion of the outer surface of the tube 76. The system may be used to confirm the placement of the feeding tube 76 in the stomach 80. The device allows the physician to distinguish different characteristics of the surrounding tissues (trachea versus esophagus 82). Specifically, the device may be used to assess the tracheal placement with the tip of the tube 84, preventing insertion of the feeding tube to a depth where patient injury is likely to occur. Also, the common technique of placing the feeding tube 76 to a safe depth and then obtaining an x-ray to confirm correct placement in the esophagus 80, followed by tube advancement and a repeat x-ray to confirm final position would reduce the number of x-rays and radiation exposure.

In another embodiment, the device may be used to provide information on the status of tissues (temperature, blood flow, oxygen levels) surrounding the tube insertion site. As well as identifying thrombus surrounding indwelling vascular catheters by build-up of pressure around the catheter over time. Also, arterial catheters such as an intra-aortic balloon pump which could have pressure sensors along length to indicate if the catheter is too large for the artery or if there is occlusion of the artery—which puts the limb at risk for ischemia. Other application would be to measure pressures in other arteries—maybe to detect cerebral or coronary artery occlusion or vasospasm. Other uses of the device may include nasogastric tubes used for decompression of the stomach, chest tubes to detect increase or persistence of a tension pneumothorax, other drainage tubes that could sense pressure build-up, or as a dermal pad.

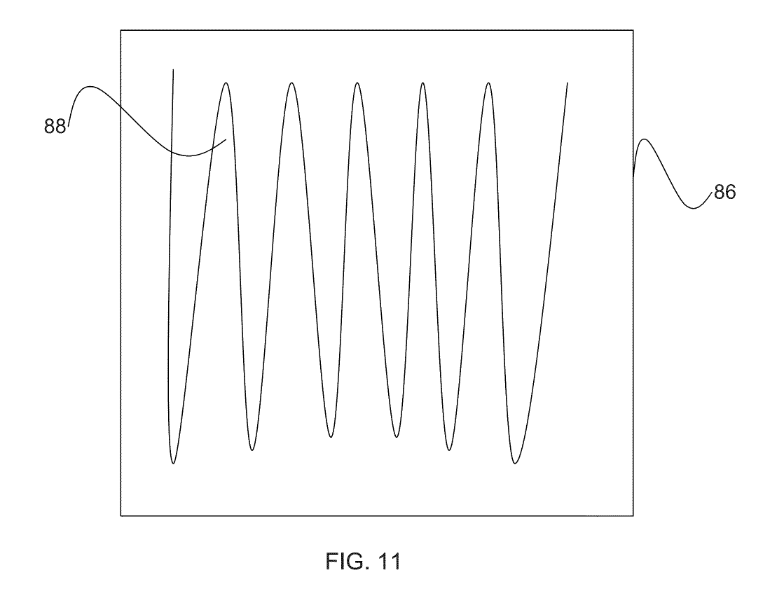

As shown in FIG. 11, in another embodiment, the system may be a dermal pad 86 or patch with external or imbedded sensors 88. The dermal pad 86 may be used to detect increased pressure on a limb that has been put into a cast. The pressure sensor 86 would detect any unusual swelling of the patient's limb, preventing unwanted pressure buildup between the limb and the cast. The dermal pad 86 may also be used to detect unwanted or undue pressure against the skin when a patient is left in one position for an extended period of time, such as for an immobilized patient or a patient in a long-term care facility to alert caregivers to skin that may be at risk of prolonged ischemia and decreasing risk of decubitis ulcer formation. The dermal pad 86 may also be used to detect unwanted or undue pressure resulting from extra-vascular fluid build-up during intra venous fluid administration such as to prevent limb compartment syndrome.

### Prophetic Example 1

A patient undergoes a cervical spine procedure that requires approaching the cervical vertebrae from the anterior neck. The surgery is uneventful but there is concern for possible airway compromise from tissue swelling due to the anterior approach. Administration of inhalational anesthesia is terminated and soon the patient exhibits their own respiratory pattern. The junior anesthesiologist performs the standard cuff leak test and appears to hear breath sounds. Noting that the blood oxygen saturation appears normal (pulse oximetry), the anesthesiologist makes the decision to extubate. Removal of the ETT is followed by choking sounds from the patient (stridor) and inability to effectively breathe.

Despite “passing” the cuff-leak test, there was a sufficient amount of swelling due to edema to close-off the upper airway. Rescue (re-)intubation fails and the patient suffocates before a tracheotomy can be performed. There are no highly sensitive or reasonably objective tests available to assure patient safety following ETT removal. The cuff-leak test is the standard but there are several different ways it can be performed, interpreted, and, in the circumstances of less experienced providers, the test may not provide the margin of safety necessary to avoid a devastating outcome.

The use of the ETT 10 would have maximized the amount of physiologic data that could have been obtained from a single invasive therapy. By using the ETT 10, the physician could detect a risk of swelling and leave the ETT 10 in place and reassess the patient at specific time intervals over the next 24 hours, possibly detecting an increase in pressure, peaking at about 24 hours then subsiding over the next 12-24 hours.

The ETT 10 tube could include CO₂ sensors 32 and pressure sensors 28 along the proximal end 14, central portion 16, and distal end 18 of the lumen 12 to assess for airway compression or soft tissue edema; a pressure sensor 28 on the outside of the inflatable cuff 20 so that the minimal amount of inflation required for sealing off the airway (minimal leak test) is applied thus reducing injury of the tracheal mucosa (an external pressure sensor 28 on the cuff 20 would also allow this evaluation and assessment of the pressure being exerted at the top of the cuff 20 to indicate if the cuff 20 is continuously in significant contact with the undersurface of the vocal cords—which predisposes to injury); and a microphone to amplify and quantify the noise of airflow around the tube during the cuff-leak test and to warn of incomplete sealing of the airway during tube placement. The microphone could also be designed for recording respiratory rate and providing auditory breath sounds. The ETT 10 may also include a tissue oximetry probe for quantification of hemoglobin oxygenation, heart rate, and total hemoglobin concentration (this would allow for monitoring of cardiovascular status and to identify times when the cuff is overinflated and thus restricting tracheal blood flow) and/or a thermal probe for recording core body temperature. The ETT 10 tube may also include other types of standard and advanced monitoring devices as technology permits, such as nitric oxide sensors in the tube lumen 12 to record changes in exhaled nitric oxide as an index of changes in systemic nitric oxide bioactivity.

### Prophetic Example 2

In another example, during intubation, the clinician may select a ETT 10 which appears to be an appropriate size for the patient based on age, gender and appearance of the airway opening. The ETT 10 may have a high volume, low pressure cuff and procedure requires checking for “minimal leak” to assure the least amount of air is in the cuff to provide a seal, thereby limiting inflation to minimal effective pressure. This is adequate at this point in time, but may change as patient conditions change or when there is prolonged intubation in the ICU. Using the ETT 10 with rapid inflation of the cuff and measuring the pressure outside the cuff after intubation may be used to assess the appropriateness of tube size and cuff inflation volume to minimize airway injury. This alerts the physician to the use of an oversized ETT 10 for the patient immediately and also serves as a baseline to compare to when checking for pressure differences at a later point in time—so as not to have excessive pressure against the vocal cords or tracheal mucosa, causing ischemia and necrosis (which may result in subsequent scarring and narrowing of the airway). This is especially applicable in pediatric patients where cuffless endotracheal tubes are often used and an oversized endotracheal tube would be detected by external sensors along the length of the endotracheal tube even without a cuff.

Moreover, the pressure sensing portion of the ETT 10 will allow non radiographic (saving on chest x-rays and radiation exposure, especially in ICU patients) representation of the ETT 10 location (a pressure footprint of tube location, coupled with CO₂ monitoring at the tip confirms location in trachea), pressure points (vocal cords, epiglottis, supraglottic soft tissue, carina, mainstem bronchi; which will change if tube position changes or edema develops), tissue oxygenation at the tracheal mucosa level and pH above the cuff to indicate aspiration of gastric content potential. In addition, the physician may also use an auditory confirmation of tube position by use of the microphone to assure intratracheal placement and continuously monitor it along with the pressure to further confirm tube position. Additionally, swallowing mechanism and other clinical issues may be evaluated with this pressure footprint.

### Prophetic Example 3

In another example, a veterinarian is completing a surgical procedure on a horse that requires a prolonged period of general anesthesia. Because horses are obligate nose-breathers when awake, the animal has two breathing tubes, one inserted through the mouth and the other inserted through one of the nares. The use of a nasal tube is appropriate especially if the horse has been in dorsal recumbency. Both tubes are outfitted with external pressure sensors. After the delivery of anesthesia is terminated, the pressure pattern generated by the mouth tube is found to be the same as the pressure pattern observed at the start of the surgery. In contrast, the nasal breathing tube displays a significant increase in pressure being exerted against the tube compared to the pressure exerted at the start of the surgery.

The veterinarian diagnoses obstruction of the nasal passages due to edema, a common occurrence in horses especially after a prolonged period of anesthesia. As the animal recovers from the anesthesia, the veterinarian removes the mouth tube but keeps the nasal breathing tube in place and uses it to provide supplemental oxygen once the horse is standing. The veterinarian next uses instillation of phenylephrine into the nasal passages to constrict the nasal mucosa. When the pressure pattern generated by the nasal breathing tube returns to normal and the horse has good movement of air through the nares, the nasal breathing tube may be removed.

Although the invention has been described with respect to a limited number of embodiments, many more variations will be readily apparent to those of skill in the art in accordance with the overall teaching and scope of this invention.

1. A system for monitoring physiologic conditions of a patient, comprising:
a medical device having an outer surface; and
a pressure sensor disposed about at least a portion of the outer surface of the medical device; wherein the pressure sensor is capable of detecting a change in pressure when a force is exerted against the outer surface of the medical device.

2. The system of claim 1, wherein the medical device is selected from the group comprising an endotracheal tube, an esophageal feeding tube, a catheter, or a dermal pad.

3. The system of claim 2, wherein the medical device is an endotracheal tube.

4. The system of claim 3, wherein the endotracheal tube comprises a lumen, the lumen having a distal end, a central portion, and a proximal end.

5. The system of claim 4, wherein the medical device further comprises an inflatable cuff disposed between the central portion of the lumen and the distal end of the lumen.

6. The system of claim 4, wherein the pressure sensor is disposed on the outer surface of the central portion of the lumen.

7. The system of claim 4, wherein the pressure sensor is disposed on the outer surface of the central portion of the lumen and the distal end of the lumen.

8. The system of claim 4, wherein the pressure sensor is disposed on the outer surface of the central portion of the lumen, the distal end of the lumen, and the inflatable cuff.

9. The system of claim 4, wherein the pressure sensor is integral with the central portion of the lumen.

10. The system of claim 4, wherein the pressure sensor is integral with the central portion of the lumen and the distal end of the lumen.

11. The system of claim 2, wherein the medical device comprises an esophageal feeding tube.

12. The system of claim 1, wherein the pressure sensor is comprised of a force sensing resistor.

13. The system of claim 1, wherein the pressure sensor is comprised of a pressure sensitive conductive polymer.

14. The system of claim 1, wherein the pressure sensor is comprised of a force-sensing biocompatible material.

15. A method for monitoring physiologic conditions of a patient, comprising:
inserting an endotracheal tube through a mouth of the patient, the endotracheal tube comprising a lumen having a proximal end, a distal end, and central portion and an inflatable cuff disposed around the lumen, between the central portion of the lumen and the distal end of the lumen, and a pressure sensor disposed about at least a portion of an outer surface of the lumen of the endotracheal tube; wherein the pressure sensor is capable of detecting a change in pressure when a force is exerted against the outer surface of the lumen;
positioning the endotracheal tube within the patient's trachea so that the patient may breath through the endotracheal tube;
taking an initial pressure reading of the pressure exerted against the pressure sensors disposed about the at least a portion of the outer surface of the lumen of the endotracheal tube; and
monitoring changes from the initial pressure reading.

16. The method of claim 15, further comprising the step of taking at least a second pressure reading.

17. The method of claim 16, further comprising the step of detecting movement of the endotracheal tube by comparing the initial pressure reading with the second pressure reading and determining if the endotracheal tube has changed positions.

18. The method of claim 16, further comprises the step of evaluating the patency of a patient's airway by comparing the initial pressure reading with the second pressure reading and determining if tissue around the endotracheal tube has swollen.

19. A method for monitoring physiologic conditions of a patient, comprising:
inserting an esophageal feeding tube through the nose or mouth of the patient, the esophageal feeding tube comprising a lumen having a proximal end, a distal end, and central portion, and a pressure sensor disposed about at least a portion of an outer surface of the lumen of the esophageal feeding tube; wherein the pressure sensor is capable of detecting a change in pressure when a force is exerted against the outer surface of the lumen;
positioning the esophageal feeding tube within the patient's esophagus;
taking an initial pressure reading of the pressure exerted against the pressure sensors disposed about the at least a portion of the outer surface of the lumen of the esophageal feeding tube; and
monitoring changes from the initial pressure reading.

20. The method of claim 19, further comprising monitoring the placement of the esophageal feeding tube within the esophagus by comparing the initial pressure reading with a known pressure pattern for a patient's esophagus.
